(19) United States
(12) Patent Application Publication
(10) Pub. No.: US 2006/0122264 A1
(43) Pub. Date: Jun. 8, 2006

(54) Stem cell invasion inhibitor

(75) Inventors: Yasumi Uchida, Funabashi-shi (JP); Haruko Uchida, Chiba (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Yasumi Uchida, etal

(21) Appl. No.: 10/559,708

(22) PCT Filed: Jun. 9, 2003
(86) PCT No.: PCT/JP03/07264

§ 371 Date: Dec. 7, 2005

(51) Int. Cl. A61K 31/365

(52) U.S. Cl. 514/462

(57) ABSTRACT

The present invention provides an inhibitor of the invasion of bone-marrow-derived vascular stem cells or inflammatory cells into blood vessel walls, a blood vessel restenosis inhibitor, and a preventive or therapeutic drug for angiitis or myocardiitis, each containing a phthalein dye compound as an active ingredient; and an instrument for treating blood vessels which is coated with a resin containing a phthalein dye compound or made of such a resin. Use of the drug of the present invention enables effective prevention of restenosis of a blood vessel after, for example, angioplasy. Moreover, it is possible to prevent the progress and disruption of a lesion of angiitis, myocarditis, or arterioscrelosis, for which no efficacious preventive or therapeutic method has been established so far, as well as microvascular ischemic diseases. By performing a medical treatment such as angioplasy using the instrument of the present invention for treating blood vessels, events of vascular restenosis can be effectively prevented.

### TECHNICAL FIELD

The present invention relates to a drug useful for preventing invasion of bone-marrow-derived vascular stem cells or inflammatory cells flowing through blood vessels into blood vessel walls or into an organ such as the heart, thereby preventing or treating restenosis following angioplasty or bypass graft surgery; and preventing or treating various vascular lesions accompanying vascular stenosis, and ischemic diseases associated therewith; and to an instrument for treatment of blood vessels (hereinafter may be referred to as a “vascular treatment instrument”) which is coated with the drug.

### BACKGROUND ART

In recent years, clinical studies and animal experiments have shown that mechanical vascular damage caused by angioplasty, bypass graft surgery, or the like thickens blood vessel walls (in particular, the intima), leading to restenosis. Hitherto, restenosis has been considered to be caused by migration of smooth muscle cells from the media into the intima, proliferation of the cells, and collagen fiber formation, and therefore clinical studies have been conducted on drugs for prevention or treatment of restenosis, including an anticoagulant, an antiplatelet drug, and a cell growth inhibitor. However, these drugs have not been shown to exhibit a satisfactory effect on restenosis.

Vascular diseases associated with hypertrophy of the intima of blood vessels or with luminal stenosis often induce hypertension, cerebral stroke, angina pectoris, myocardial infarction, or necrosis of extremities. Examples of such vascular diseases known heretofore include Kawasaki disease, aortitis syndrome, and arteriosclerotic lesion (plaque).

Kawasaki disease, which is a tragic inflammatory proliferative vascular disease, often occurs in Japanese infants or children, and is associated with a high incidence of coronary artery aneurysm, which may be followed by stenosis, resulting in angina pectoris or myocardial infarction (Kawasaki T: Kawasaki Disease Nankoudou Co, Tokyo, 1988, pp. 68-70; Kato H: J Pediatr 1986, 108: 923-928). Conceivably, coronary artery aneurysm is caused by destruction of blood vessel walls due to inflammation, and stenosis is caused by highly advanced intimal fibrous hyperplasia (Uchida Y: Coronary Angioscopy, Futura Publishing Co, NY, 2000, pp. 125-129; Ishikawa H: Circulation 1991, 84 (Suppl II): 15). However, an effective method of preventing or treating this disease has not yet been known.

Aortitis syndrome, which is an inflammatory proliferative vascular disease, is frequently found in Japanese women, but a method for prevention or radical treatment of this disease has not yet been developed. Angiitis occurs in most large blood vessels, including the coronary artery, aorta, pulmonary artery, cerebral artery, and carotid artery, and causes stenosis of these blood vessels. Inflammation occurs primarily in the adventitia, which results in intimal hyperplasia. However, the mechanism of intimal hyperplasia or stenosis has not been elucidated (Ishikawa K: Circulation 1978, 57: 27-30), and thus an effective method for preventing or treating this disease remains unknown.

Examples of angiitis similar to those described above include giant cell arteritis, and arteritis associated with contractural arthritis (Barkley B H: Circulation 1973, 43: 1014).

Arteriosclerotic lesion (plaque) is considered to be associated with stenosis resulting from intimal fibrous hyperplasia and lipid deposition, and causes angina pectoris, myocardial infarction, cerebral infarction, or obstructive peripheral arteriopathy.

Angina pectoris due to coronary microcirculation disorder often occurs in menopausal women, and this disease has been elucidated to be associated with proliferation of smooth muscle cells in the media of coronary arteriole and hyperplasia of the media, or with endothelial dysfunction (Cannon ROI: Am J Cardiol, 1988, 61: 1335-1349; Mohri M: Lancet, 1998, 351: 1165-1169; Rosono J M: J Am Coll Cardiol, 1996, 28: 1500-1505). However, the mechanism of proliferation of smooth muscle cells in the media or hyperplasia of the media has not been elucidated, and a method of preventing or treating this disease has not yet been established.

As has been well known, inflammatory cells (e.g., macrophages or dendritic cells), which are also bone-marrow-derived differentiated cells, invade blood vessel walls and are involved in progression of arteriosclerosis. A recent study has shown that vascular stem cells present in circulating blood migrate from the lumen into the intima, and cause restenosis or arteriosclerotic lesion after angioplasty or bypass graft surgery (Sata M: Nature Medicine 2002, 8(4): 403-409). Another recent study has shown that invasion of vascular stem cells is involved in restenosis after artificial vascular grafting (Shimizu K: Nature Medicine 2001, 7: 738-741). The present inventors have found that vascular stem cells are positive for β-actin. Identification of vascular stem cells is based on that the stem cells are also positive for CD34, factor 8, vimentin, or the like, and have the ability to be differentiated into various cell types constituting blood vessels (Yasumi Uchida: “Kekkan” (“Blood Vessel”) 2002, 26: 22). The present inventors have also found that vascular stem cells are positive for GFAP (glial fibrillary acid protein); i.e., vascular stem cells are neural stem cells, and that vascular stem cells also invade blood vessel walls or myocardium. Furthermore, the present inventors have reported that restenosis after angioplasty by use of a balloon or a stent occurs by the following steps: 1) vascular stem cells migrate from the vascular lumen into the intima; 2) vascular stem cells migrate outward from capillary vessels present on the adventitia, followed by invasion of the cells through the adventitia, the media, and the internal elastic lamina, into the intima; and 3) vascular stem cells migrate from a new blood vessel formed on the thickened intima, become positive for α-actin, and are formed into collagen fibers. The present inventors have also reported that invasion of vascular stem cells through the adventitia in step 1) plays an important role in the development of restenosis (Uchida Y, et al.: Circulation J, 2002, 66 (Suppl I): 273).

It has been suggested that arteriosclerosis is associated with inflammation, and is also associated with inflammatory cells differentiated from bone-marrow-derived stem cells (Ehara S: Ciculation J 2002, 66 (Suppl I): 142; Libby P: Circulation J 2002, 66 (Suppl I): 32-33). The present inventors have also found that, in a dog model of angiitis, invasion of β-actin-positive stem cells through the adventitia into the intima induces intimal hyperplasia (Japanese Patent Application Laid-Open (kokai) No. 2003-79647). Furthermore, the present inventors have succeeded in developing a dog model of coronary microvascular ischemia, and have found that marked proliferation of smooth muscle cells in the media of the arteriole of this model—such cell proliferation is also observed in clinical studies—is caused by invasion of β-actin-positive stem cells through the adventitia.

Thus, recent studies have shown that vascular restenosis and vascular diseases accompanying vascular hyperplasia or stenosis are intimately related to invasion of vascular stem cells or inflammatory cells into blood vessel walls.

An object of the present invention is to provide a drug useful for preventing invasion of vascular stem cells or inflammatory cells into blood vessel walls or into an organ such as the heart, thereby preventing or treating various vascular lesions associated with vascular stenosis caused by inflammation or restenosis following angioplasty or bypass graft surgery.

### DISCLOSURE OF THE INVENTION

In view of the foregoing, the present inventors have searched for a compound which readily permeates the adventitia of blood vessels and which exhibits the effect of preventing invasion of vascular stem cells or inflammatory cells into blood vessel walls, and as a result have found that a phthalein dye compound, which is currently employed as a clinical test reagent, exhibits the effect of inhibiting invasion of vascular stem cells, dendritic cells, or the like into blood vessel walls, thereby inhibiting stenosis or restenosis. The present inventors have also found that when an instrument (e.g., a drug-eluting stent) is coated with a resin containing such a dye compound, or is prepared from the resin, and the resultant instrument is inserted into blood vessels, restenosis or the like can be effectively prevented.

Accordingly, the present invention provides a drug for preventing invasion of bone-marrow-derived vascular stem cells or inflammatory cells into blood vessel walls, the drug containing a phthalein dye compound as an active ingredient.

The present invention also provides a drug for preventing vascular restenosis, a preventive or therapeutic drug for angiitis or myocarditis, a preventive or therapeutic drug for a microvascular ischemic cardiac disease, a preventive or therapeutic drug for an angiogenic disease, a preventive or therapeutic drug for a proliferative disease caused by cells derived from neural stem cells, a drug for preventing graft rejection, and a drug for preventing arteriosclerosis associated with organ transplantation, each of the drugs containing the aforementioned dye compound as an active ingredient.

The present invention also provides a vascular treatment instrument coated with a resin containing the aforementioned dye compound.

The present invention also provides a vascular treatment instrument prepared from a resin containing the aforementioned dye compound.

The present invention also provides a percutaneous transluminal arteriovenous angioplasty or an arteriovenous bypass graft surgery, characterized by employing the aforementioned vascular treatment instrument.

The present invention also provides a method of preventing vascular restenosis, characterized by comprising subjecting a patient to a medical treatment employing the aforementioned vascular treatment instrument.

The present invention also provides a method of treating an inflammatory angiocardiac disease, characterized by comprising administering the aforementioned dye compound to a patient in need thereof.

### BRIEF DESCRIPTION OF THE DRAWINGS

FIG. 1 shows the effect of phenolsulfonphthalein in preventing cell invasion associated with dog coronary angioplasty by use of a balloon (one week after angioplasty, azan staining, ×400, scale: 10 μm):

(A): site at which angioplasty was not performed, M: media, A: adventitia, arrowhead: internal elastic lamina;

(B): site at which angioplasty was performed, M: media, A: adventitia, arrows: numerous invaded cells; and

(C): site at which angioplasty was performed and then phenolsulfonphthalein was administered, M: media, A: adventitia, virtually no cell invasion is observed.

FIG. 2 shows the effect of phenolsulfonphthalein in preventing stenosis due to intimal hyperplasia associated with coronary angioplasty by use of a balloon (four weeks after angioplasty, azan staining, ×400):

(A): non-administration of phenolsulfonphthalein, M: media, A: adventitia, I: thickened intima, arrow: internal elastic lamina; and

(B): administration of phenolsulfonphthalein, M: media, A: adventitia, arrow: internal elastic lamina, only slight intimal hyperplasia.

FIG. 3 shows images of various immunostained vascular stem cells in thickened intima in the case of non-administration of phenolsulfonphthalein (×1,000):

A: CD34-positive cells; i.e., bone-marrow-derived cells;

B: GFAP-positive cells; i.e., neural stem cells;

C: vimentin-positive cells; i.e., fibroblasts;

E to G: β-SM-actin-positive cells; i.e., immature smooth muscle cells; and

H: α-SM-actin-positive cells; i.e., matured smooth muscle cells.

FIG. 4 shows a stent formed from a photopolymerizable resin containing phenolsulfonphthalein (scale: 1 mm):

A: 2: stent having an inner diameter of 0.8 mm; and

B: dilation balloon catheter covered with the stent (1: balloon catheter, 3: guide wire).

FIG. 5 is a schematic representation showing a pored balloon catheter.

1: Pressure-applying means

2: Drug-supplying means

3: Puncture needle

4: Connecting means

5: Balloon

6: Pores

7: Guide wire

### BEST MODE FOR CARRYING OUT THE INVENTION

Examples of the phthalein dye compound, which is an active ingredient of the drug of the present invention, include phenolsulfonphthalein, bromosulfophthalein, and phenolphthalein. Of these, particularly preferred are phenolsulfonphthalein, which is routinely employed as a clinical reagent for testing renal excretory function, and bromosulfophthalein, which is routinely employed as a clinical reagent for testing hepatic excretory function.

As described below in Examples, such a dye compound employed in the present invention exhibits the effect of inhibiting invasion of bone-marrow-derived vascular stem cells or inflammatory cells (e.g., dendritic cells) into blood vessel walls (adventitia, media, and intima)—such invasion is caused by mechanical vascular damage or inflammation—thereby inhibiting vascular stenosis or restenosis. Therefore, the dye compound employed in the present invention can serve as a drug for preventing invasion of bone-marrow-derived vascular stem cells or inflammatory cells into blood vessel walls. Examples of the bone-marrow-derived inflammatory cells include dendritic cells and macrophages.

Administration of the dye compound exhibiting the aforementioned effect inhibits invasion of stem cells into the intima, destruction of the media upon such invasion, transformation of smooth muscle cells into collagen fibers, intimal hyperplasia, as well as neovascularization. Therefore, the dye compound employed in the present invention can serve as a drug for effectively preventing intimal hyperplasia or vascular restenosis caused by intimal hyperplasia, which is associated with angioplasty, bypass graft surgery, or the like. In addition, the dye compound is useful as a preventive or therapeutic drug for vascular proliferative diseases caused by, for example, inflammatory, metabolic, or congenital proliferation-promoting vascularization, including proliferative retinopathy due to hypervascularization by collagen fibers, exacerbation of congenital arteriovenous fistula, or promotion of proliferation of solid tumors through nutrient vessel formation. For example, the dye compound can serve as a preventive or therapeutic drug for angiitis, such as Kawasaki disease, aortitis syndrome, giant cell arteritis, or arteritis associated with ankylosing arthritis; arteriosclerotic lesion (plaque); myocarditis; microvascular ischemic cardiac diseases including coronary microcirculation disorder; or small vessel-related diseases such as moyamoya disease and diabetic retinopathy.

Stem cell invasion has been reported to be involved in sclerosis of grafted blood vessels (Saihara A: Nature Medicine, 2001, 7(4): 382-383). Also, stem cells are considered to be involved in rejection associated with organ transplantation. Therefore, the dye compound employed in the present invention can serve as a drug for preventing graft rejection associated with organ transplantation, and as a preventive or therapeutic drug for arteriosclerosis associated with organ transplantation.

Meanwhile, as described above, vascular stem cells are positive for GFAP (glial fibrillary acid protein); i.e., vascular stem cells are neural stem cells. Therefore, the dye compound employed in the present invention can serve as a drug for treating a proliferative disease caused by cells derived from neural stem cells such as glioma.

In the drug of the present invention, phthalein dye compounds may be employed singly or in combination of two or more species.

When the dye compound employed in the present invention is used in combination with a sulfonic acid-based azo dye containing a group —SO₃⁻ in the molecule (e.g., Evans Blue, Trypan Blue, or Trypan Red), which is considered to exhibit a pharmacological effect similar to that of the dye compound, the effect of the dye compound in preventing invasion of vascular stem cells, etc. into blood vessel walls is enhanced, which is preferred.

The ratio by weight of the dye compound employed in the present invention to such a sulfonic acid-based azo dye is preferably 1:0.1 to 1:100, particularly preferably 1:0.5 to 1:1.

When the dye compound is employed in combination with, for example, a known immunosuppressive drug, immunomodulator, collagen synthesis inhibitor, or anticancer drug, the effect of preventing invasion of vascular stem cells, etc. into blood vessel walls can be further enhanced. Examples of the immunosuppressive drug to be employed include sirolimus, paclitaxel, tacrolimus, azathioprine, cyclosporine, cyclophosphamide, mycophenolate mofetil, gusperimus, mizoribine, tranilast, and pemirolast. Of these, sirolimus, paclitaxel, and tacrolimus are preferred. Examples of the immunoregulator include leflunomide and interferon. Examples of the collagen synthesis inhibitor include halfginone. Examples of the anticancer drug include actinomycin.

The ratio by weight of the dye compound employed in the present invention to the aforementioned immunosuppressive drug is preferably 1:0.01 to 1:10, particularly preferably 1:0.1 to 1:1.

In the case where the dye compound is employed in combination with the aforementioned drug, a single preparation may be formed so as to contain both of them, or different preparations may be formed from the dye compound and the drug, and the preparations may be used in combination.

The drug of the present invention may be formulated together with a pharmaceutically acceptable carrier into a pharmaceutical composition for parenteral administration (e.g., injection or transrectal administration), or a solid or liquid pharmaceutical composition for oral administration.

The pharmaceutical composition for injection may be in the form of a solution (sterile aqueous or non-aqueous solution), an emulsion, or a suspension. Examples of the non-aqueous carrier, diluent, solvent, or vehicle to be employed in the composition include propylene glycol, polyethylene glycol, vegetable oils such as olive oil, and injectable organic acid esters such as ethyl oleate. The composition may appropriately contain an auxiliary agent such as an antiseptic, a humectant, an emulsifier, or a dispersant.

The composition for oral administration may be in a solid form, such as a tablet, a powder, a granule, or a capsule; or in a liquid form, such as a solution, a syrup, an elixir, or an oil or aqueous suspension.

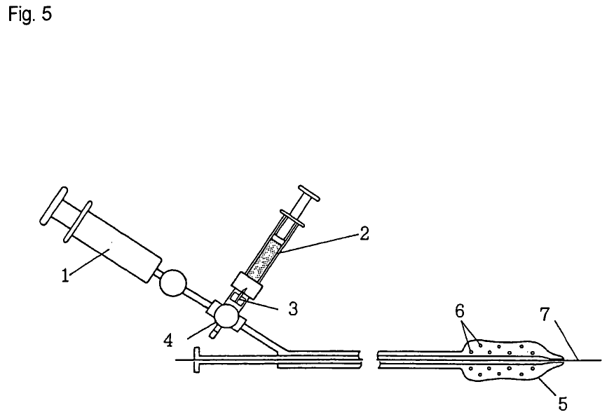

Among the aforementioned administration forms, administration in the form of an injection is preferred for the drug of the present invention. More preferred are, for example, intravenous administration, intraarterial administration, intragraft local administration, and pressurized local administration by use of a perforated balloon catheter. Particularly preferred is administration by use of a pored balloon catheter (Japanese Patent Application No. 2002-190235) as shown in FIG. 5.

The dose of the drug of the present invention varies depending on the properties of ingredients to be administered, the administration route, the desired treatment period, and other factors. The daily dose of the drug is typically about 0.1 to about 100 mg/kg, particularly preferably about 0.2 to about 10 mg/kg, as reduced to the dye ingredient. If desired, the daily dose may be administered in a divided manner (2 to 4 times a day).

The vascular treatment instrument of the present invention may be formed by coating the surface of a treatment instrument which is generally employed for angioplasty with a resin containing one or more phthalein dye compounds (hereinafter the resin may be referred to simply as a “dye-containing resin”), or may be formed from the dye-containing resin serving as a raw material.

No particular limitation is imposed on the resin to be employed in the present invention, so long as the resin has malleability, flexibility, and moisture permeability, is suitable for coating formation, and causes no problems when used in a living organism. The resin may be a biodegradable resin, a hard-to-biodegrade resin, or a mixture of these resins. Examples of the hard-to-biodegrade resin include polyolefin polymers (e.g., polyethylene, polypropylene, polybutene, polyisoprene, SEBS, SIS, and polyolefin copolymers), acrylic resins (e.g., polyacrylic acid, polyacrylic acid salts, and polybutyl acrylate), fluorocarbon reins (e.g., polytetrafluoroethylene), polystyrene, polyvinyl chloride, polyurethane, polyamide, polyester, cellulose, polycarbonate, nylon, silicone resin, natural rubber latex, polyvinyl alcohol, polyprolactone, and polyvinyl acetamide. Examples of the biodegradable resin include polyglycolic acid, polylactic acid, polylactic acid/polyglycolic acid copolymers, polyphosphoric acid, polyglutamic acid, collagen, gelatin, glucosaminoglycan, hyaluronic acid, fibrin, alginic acid, chitin, chitosan, fibroin, and salts thereof. Of these, particularly preferred are polyglycolic acid, polylactic acid, polyphosphoric acid, or a mixture of such a material and polyisoprene or the like.

No particular limitation is imposed on the material or form of the treatment instrument with which the aforementioned resin is to be coated, so long as the treatment instrument can be employed for treatment of blood vessels, and placed in blood vessels. The material of the treatment instrument may be a biodegradable (self-absorbable) material or a hard-to-biodegrade material. Examples of the material include hard-to-biodegrade materials such as stainless steel, nickel-titanium alloy, metal (e.g., tantalum), ceramic material, polytetrafluoroethylene, polytrifluoroethylene, polyethylene, polyethylene terephthalate, polypropylene, carbon nanosheet, and carbon nanotube; and biodegradable materials such as polylactic acid, polyphosphoric acid, polyglycolic acid, polymalic acid, and poly(α-amino acid).

The treatment instrument may be formed of a photopolymerizable material prepared by mixing the aforementioned material with a photopolymerizable resin (e.g., urethane dimethacrylate). In the case where such a photopolymerizable material is employed, when the instrument is inserted into a blood vessel, and then irradiated with light having a specific wavelength, the photopolymerizable resin is cured, and thus the blood vessel lumen can be dilated.

No particular limitation is imposed on the form of the treatment instrument, so long as the instrument has a strength such that it can be reliably placed on the inner wall of a blood vessel. Preferably, the treatment instrument is formed of, for example, a cylindrical mesh structure.

Specific examples of the vascular treatment instrument of the present invention include a stent, a stent graft, a catheter, a balloon, and an artificial blood vessel. Of these, a stent and an artificial blood vessel are particularly preferred. Such a stent may have a coil form or a cylindrical mesh form, and the stent may be a rigid stent or a flexible stent.

Coating the aforementioned treatment instrument with a dye-containing resin is typically carried out through the following procedure: a resin is dissolved in an organic solvent (e.g., an alcohol, an ether, dichloromethane, cyclohexane, dimethylformamide, or dimethyl sulfoxide); a dye compound is mixed with the resultant solution, and then dissolved therein at room temperature or, if desired, under heating, to thereby prepare a dye-containing resin solution; and the treatment instrument is coated with the dye-containing resin solution by immersing the instrument in the solution or by spraying the solution onto the instrument, followed by drying and sterilization. In order to achieve release of the dye compound immediately after insertion of the treatment instrument into a blood vessel, as well as long-term sustained-release of the dye compound, preferably, the above-coated treatment instrument is further coated with a dye compound solution or a dye-containing polymer solution, thereby attaining multi-layer coating of the instrument. For example, in the case of two-layer coating, firstly, the treatment instrument is coated with a dye-containing resin solution containing, as a resin, a polylactic acid/polyglycolic acid copolymer (e.g., PLGA 7510 (Wako)), thereby forming a sustained-release layer (inner layer), and subsequently, the resultant instrument is further coated with a dye-containing resin solution containing, as a resin, polylactic acid (e.g., PLA 0010 (Wako)), thereby forming a rapid-release layer (outer layer). In the case of three-layer coating, preferably, the inner layer is formed from a dye-containing resin solution containing, as a resin, a polylactic acid/polyglycolic acid copolymer (e.g., PLGA 7510 (Wako)); the intermediate layer is formed from a dye-containing resin solution containing polybutyl acrylate; and the outer layer is formed from a dye-containing resin solution containing polylactic acid (e.g., PLA 0010 (Wako)) (as used herein, “inner layer” refers to a layer which is in contact with the treatment instrument (e.g., a stent)).

The dye compound content of the dye-containing resin solution is preferably 0.5 to 50%, particularly preferably 10 to 20%, from the viewpoint of the restenosis-preventing effect.

In the case where the treatment instrument per se is to be formed from a dye-containing resin (for example, in the case of formation of a stent), a tube (e.g., a polyethylene tube) is immersed in the aforementioned dye-containing resin solution, and then the resin is cured, followed by removal of the tube from the resultant product. Preferably, the resin solution to be employed is mixed with a photopolymerizable resin (e.g., urethane dimethacrylate).

When the thus-formed vascular treatment instrument of the present invention is employed for a medical treatment such as a percutaneous transluminal angioplasty (e.g., coronary angioplasty, peripheral angioplasty, or cerebral angioplasty) or an arteriovenous bypass graft surgery (e.g., coronary bypass graft surgery or peripheral artery bypass graft surgery), vascular restenosis, which would otherwise occur following the medical treatment, can be effectively prevented.

### EXAMPLES

### Example 1

### Local Administration Test by Use of a Pored Balloon Catheter Connected to a Drug Injector

Phenolsulfonphthalein or bromosulfophthalein exhibits very high tissue affinity, and, when such a phthalein dye compound adheres to the skin, clothing, or a medical instrument during handling of the compound, the compound is very difficult to remove therefrom. Therefore, when such a phthalein dye compound is employed clinically, leakage of the compound to the outside must be prevented. Thus, administration of such a dye compound requires a closed-circuit administration apparatus for preventing leakage of the compound to the outside. In view of the foregoing, in this Example, there was employed a pored balloon catheter which percutaneously enables such a phthalein dye compound to be selectively administered to a local site in blood vessels such that the catheter is not exposed to the outside, which catheter has previously been applied for a patent (see FIG. 5, Japanese Patent Application No. 2002-190235).

(A) Test on Prevention of Stem Cell Invasion Associated with Coronary Angioplasty

(1) Test Method

Nembutal-anesthetized beagle dogs were employed for the test. Each of the animals was endotracheally intubated, and ventilated with air. Subsequently, an 8-French sheath was inserted into the right common carotid artery. An 8-French guide catheter was inserted through the sheath, followed by coronary angiography. Subsequently, a clinically employed balloon catheter for coronary angioplasty (outer diameter: 3 mm, length: 20 mm) was inserted, and then the balloon was expanded at 9 atm, to thereby dilate a distal to intermediate portion of the anterior descending branch of the coronary artery. Through this procedure, the blood vessel wall was damaged. Subsequently, the aforementioned perforated balloon catheter equipped with an injection syringe containing 5% phenolsulfonphthalein (0.5 mL) or 5% bromosulfophthalein (0.5 mL) was guided by a guide wire and inserted into the above-dilated portion, and the phthalein compound (drug) was injected into the blood vessel wall at 3 atm. After completion of injection of the drug, the catheter was removed, and then the cervical portion through which the catheter had been inserted was sutured, followed by administration of an antibiotic. After recovery from anesthesia, the animal was cared for one week to one month, and was again subjected to coronary angiography. One week to one month later, the animal was anesthetized to stop cardiac beat and, the heart was removed from the animal, and then subjected to formalin fixation, followed by preparation of a microscopic sample of the above-dilated portion. Subsequently, the thus-prepared sample was subjected to azan staining and various immunostainings.

The present inventors have already found that stem cells in circulating blood are positive for vascular smooth muscle β-actin, and are intimately involved in restenosis following angioplasty (Uchida Y: Circulation J 2002, 66 (Suppl 1): 273). In connection with this finding, in the above-immunostained sample, stem cells per unit area (250×250 um²) were counted at the highest-cell-density site of the intima, media, or adventitia. Comparison (in terms of the number of stem cells) was performed between drug administration groups and a non-administration group (control group) in which merely saline (0.5 mL) was injected into a portion of the coronary artery dilated with a balloon, and P<0.05 (by Student t test) was regarded as a significant difference.

(2) Results

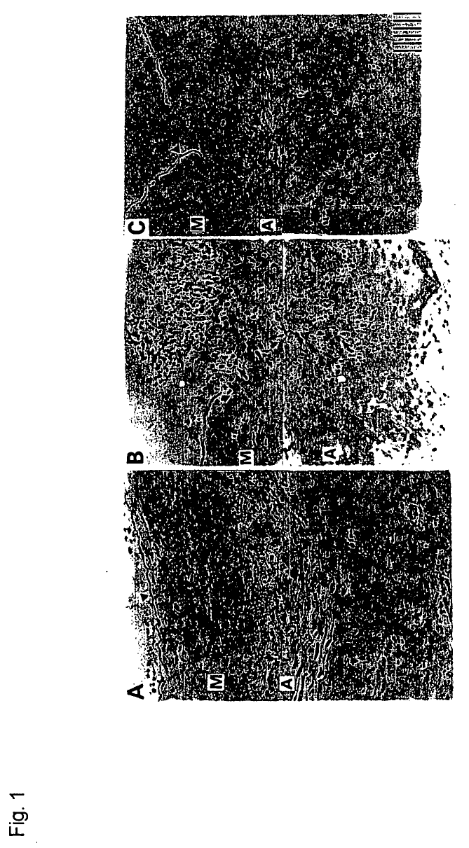

FIG. 1 shows coronary artery cross sections one week after dilation with a balloon. In the control sample, invasion of numerous cells from the adventitia into the intima is observed. In contrast, in the phenolsulfonphthalein local administration sample, such cell invasion is not observed.

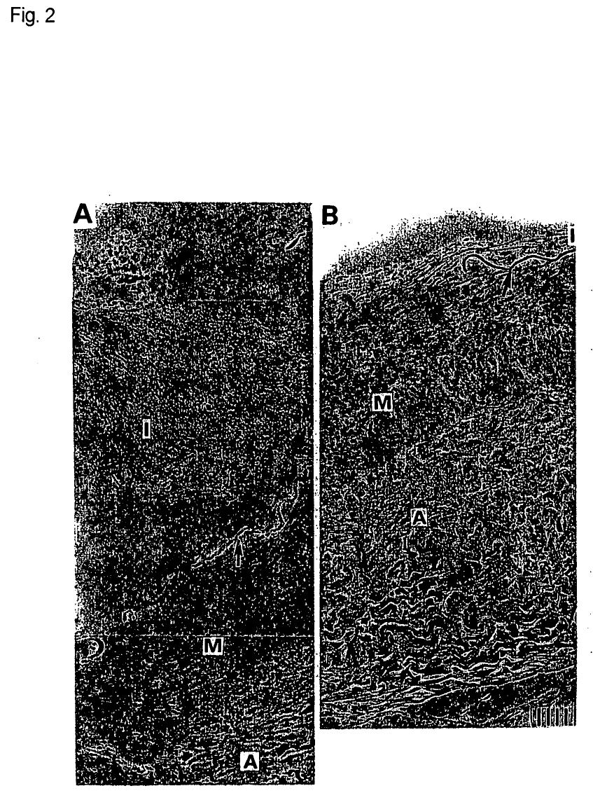

FIG. 2 shows coronary artery cross-sectional images four weeks after dilation. In the control sample, stenosis due to intimal hyperplasia is markedly observed. In contrast, in the phenolsulfonphthalein administration sample, stenosis due to intimal hyperplasia is only slightly observed, and cell invasion is less observed.

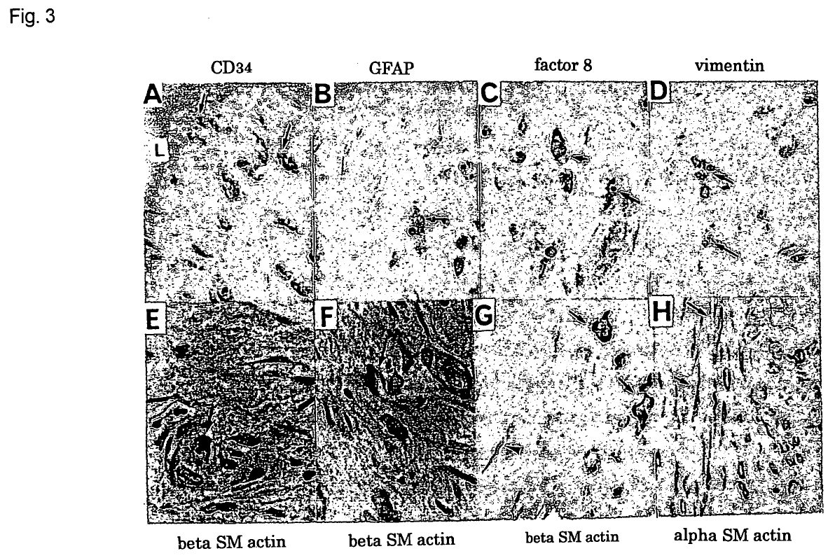

FIG. 3 shows invasion of CD34-positive cells (i.e., bone-marrow-derived cells) or β-actin-positive cells (i.e., immature smooth muscle cells) from the adventitia or new blood vessel into the intima.

Table 1 shows the density of stem cells at the intima, media, and adventitia of the coronary artery.

In the non-administration group, the stem cell density is highest at the adventitia. The results indicate that invasion of stem cells from the adventitia into the intima plays an important role for stenosis. In the phenolsulfonphthalein administration group or the bromosulfophthalein administration group, at all the membranes, the stem cell density is significantly lower than that in the case of the non-administration group. The results indicate that these drugs have the effect of inhibiting cell invasion.

Dendritic cells, which are inflammatory cells, were subjected to immunostaining with a CD40 antibody. Table 1 shows the results, which indicate invasion of dendritic cells is inhibited by phenolsulfonphthalein or bromosulfophthalein.

Table 2 shows the degree of stenosis as determined through coronary angiography. In the phenolsulfonphthalein administration group or the bromosulfophthalein administration group, stenosis is significantly inhibited.

TABLE 1
Change in the number of β-actin-positive stem cells or dendritic cells
in the coronary artery wall through local administration of a drug

| | | | Number of |
|---|---|---|---|
| | | | dendritic |
| | Number of | Number of | cells (250 × |
| | samples | stem cells | 250 μm²) |
| Intima | | | |
| Non-administration group | 6 | 28.5 ± 6.0 | 8.3 ± 1.2 |
| Phenolsulfonphthalein group | 6 | 11.0 ± 4.2* | 0.2 ± 0.0* |
| Bromosulfophthalein group | 6 | 14.8 ± 6.1* | 1.6 ± 0.4* |
| Media | | | |
| Non-administration group | 6 | 22.0 ± 5.2 | 4.3 ± 1.0 |
| Phenolsulfonphthalein group | 6 | 10.1 ± 2.8* | 0.0 ± 0.0* |
| Bromosulfophthalein group | 6 | 16.1 ± 4.8 | 1.1 ± 0.1 |
| Adventitia | | | |
| Non-administration group | 6 | 38.8 ± 12.0 | 10.4 ± 2.2 |
| Phenolsulfonphthalein group | 6 | 19.3 ± 6.3* | 1.0 ± 0.5* |
| Bromosulfophthalein group | 5 | 22.0 ± 6.2* | 3.6 ± 0.9* |

Comparison with non-administration group:
*P < 0.05,
**P < 0.01,
***P < 0.001

TABLE 2
Change in coronary artery stenosis through administration of a drug

| | Number of | Stenosis |
|---|---|---|
| | samples | degree (%) |
| Non-administration group | 6 | 79.2 ± 19.6 |
| Phenolsulfonphthalein group | 6 | 10.1 ± 2.5** |
| Bromosulfophthalein group | 5 | 33.5 ± 10.4* |

(B) Test on Inflammatory Stenosis Caused by Administration of a Foreign Substance to the Coronary Artery Adventitia
(1) Test Method

Anesthetized beagle dogs were employed for the test. While each of the animals was ventilated with air, thoracotomy was performed at the left fifth intercostal space, to thereby expose the heart. Subsequently, talc powder was administered to the vicinity of the adventitia at an intermediate portion of the anterior descending branch of the left coronary artery, which caused inflammation. Subsequently, the thoracotomy site was closed, and phenolsulfonphthalein was injected under conditions similar to those of the test described above in (A) by use of a perforated balloon equipped with an injection syringe containing the drug. After recovery from anesthesia, the animal was bred for one month, and then subjected to coronary angiography. Thereafter, the heart was extirpated from the animal, and then subjected to formalin fixation. A microscopic sample of the talc-administered portion was prepared, and the sample was subjected to β-actin staining, followed by determination of the density of β-actin-positive cells (i.e., stem cells).

(2) Results

Table 3 shows the density of β-actin-positive cells. In the phenolsulfonphthalein administration group, the β-actin-positive cell density is significantly lower than that in the case of the non-administration group. As shown in Table 4, the degree of stenosis is significantly lower in the drug administration group.

TABLE 3
Density of β-actin-positive stem cells at the talc-administered site

| | Number of | Number of stem cells |
|---|---|---|
| | samples | (250 × 250 μm²) |
| Intima | | |
| Non-administration group | 5 | 11.6 ± 2.3 |
| Phenolsulfonphthalein | 5 | 2.0 ± 0.8* |
| administration group | | |
| Media | | |
| Non-administration group | 5 | 19.9 ± 3.4 |
| Phenolsulfonphthalein | 5 | 2.6 ± 0.7* |
| administration group | | |
| Adventitia | | |
| Non-administration group | 5 | 80.6 ± 11.9 |
| Phenolsulfonphthalein | 5 | 12.2 ± 3.4* |
| administration group | | |

TABLE 4
Degree of stenosis at the talc-administered site

| | Number of | |
|---|---|---|
| | samples | Stenosis degree (%) |
| Non-administration group | 5 | 42.5 ± 10.1 |
| Drug administration group | 5 | 10.4 ± 2.7* |

### Example 2

### Effect of Phenolsulfonphthalein or Bromosulfophthalein in Inhibiting Proliferation of Human Vascular Smooth Muscle Cells, as Well as Antithrombotic Effect of the Drug

The present inventors have demonstrated that, by use of human coronary artery smooth muscle cells, Evans Blue has the effect of inhibiting proliferation of vascular smooth muscle cells, as well as antithrombotic effect (International Publication WO 01/93871 pamphlet), and that these effects are attributed to inhibition of the G1 phase of the cell cycle (the same pamphlet). In connection therewith, phenolsulfonphthalein or bromosulfophthalein was tested to determine whether or not it has such effects.

(A) Effect on Proliferation of Vascular Smooth Muscle Cells or Endothelial Cells

(1) Test Method

Human umbilical vein endothelial cells (HUBEC) (1×10⁵/cm²) were inoculated into a medium containing MCDB-104, 10% FBS, ECGF (100 ng/mL), EGF (10 ng/mL), and heparin (100 ng/mL), and phenolsulfonphthalein or bromosulfophthalein was added to the medium, followed by culturing for three days. Thereafter, the resultant cells were counted, and the number of the cells was compared with that in the case where no drug was added to the medium. In a manner similar to that described above, human coronary artery smooth muscle cells (1×10⁵/cm²) were cultured in a medium containing MCDB-104, 10% FBS, and EGF (10 ng/mL) for three days, and the resultant cells were counted.

(2) Results

As shown in Table 5, phenolsulfonphthalein or bromosulfophthalein exhibits no effect of inhibiting proliferation of human vascular endothelial cells (HUBEC) or vascular smooth muscle cells.

TABLE 5
Effect of phenolsulfonphthalein, bromosulfophthalein, or
Evans Blue in inhibiting proliferation of human coronary
artery smooth muscle cells or vascular endothelial cells

| | Concentration (mol) | | | | | |
|---|---|---|---|---|---|---|
| | Control | 10⁻⁸ | 10⁻⁷ | 10⁻⁶ | 10⁻⁵ | 10⁻⁴ |
| Smooth muscle cells | | | | | | |
| Phenolsulfonphthalein | 3.81 | 4.08 | 4.02 | 4.17 | 3.88 | 3.65 |
| Bromosulfophthalein | 4.05 | 4.24 | 4.14 | 4.19 | 3.78 | 3.43 |
| Evans Blue | 4.01 | 3.89 | 3.50* | 1.00* | 0.34** | 0.07*** |
| Endothelial cells | | | | | | |
| Phenolsulfonphthalein | 3.35 | 3.47 | 3.43 | 3.35 | 3.40 | 3.37 |
| Bromosulfophthalein | 3.50 | 3.34 | 3.59 | 3.52 | 3.43 | 3.44 |
| Evans Blue | 3.66 | 3.50 | 3.11* | 1.54* | 0.98* | 0.12** |

*P < 0.05,
**P < 0.01 against control

(B) Effect on Arterial Thrombosis
(1) Test Method

Anesthetized dogs were employed for the test. Phenolsulfonphthalein or bromosulfophthalein (10 mg/kg, which is 100 times the amount clinically used for renal function testing) was intravenously administered to each of the animals, and the intima of the right common carotid artery was damaged by means of a Dotter balloon catheter. Thirty minutes later, the common carotid artery was extirpated, and the wet weight of thrombus present in the carotid artery was measured. The thus-measured wet weight was compared with that in the case of the control group.

(2) Results

As shown in Table 6, phenolsulfonphthalein or bromosulfophthalein exhibits no effect of inhibiting thrombus formation.

TABLE 6
Effect of phenolsulfonphthalein or bromosulfophthalein
in inhibiting thrombus formation

| | Number of | Average thrombus |
|---|---|---|
| | samples | weight (mg) |
| Control group | 4 | 230 |
| Phenolsulfonphthalein group | 4 | 195 |
| Bromosulfophthalein group | 4 | 210 |

The above results reveal that phenolsulfonphthalein or bromosulfophthalein has neither the effect of inhibiting proliferation of vascular smooth muscle cells, nor antithrombotic effect.

Therefore, conceivably, the mechanism by which such a drug prevents restenosis is not associated with the effect of inhibiting proliferation of vascular smooth muscle cells nor with antithrombotic effect, but with the effect of inhibiting invasion/migration of stem cells.

### Example 3

### Preparation of Drug-eluting Stent by Use of Phenolsulfonphthalein-containing Resin

As has been shown, invasion of stem cells into blood vessel walls continues for four weeks or more. Therefore, in order to completely prevent such stem cell invasion, there must be created a mechanism enabling a drug to be released to a local site in a sustained manner for four weeks or more. In view of the foregoing, attempts were made to develop a stent having such a mechanism, which is to be locally placed.

(A) Stent Coated with Drug-containing Resin

(1) Singly-layer-coated Drug-eluting Stent

Resin: PLA 0010 (polylactic acid), PLGA 5010 or PLGA 7510 (polylactic acid/polyglycolic acid copolymer), polyvinyl alcohol, polyprolactone, polyglycolic acid, polyvinyl acetamide, gelatin, or hyaluronic acid;

Drug: phenolsulfonphthalein, bromosulfophthalein, or a mixture of phenolsulfonphthalein and Evans Blue;

Solvent: dichloromethane, cyclohexane, water, or ethanol; and

Stent: commercially available coronary stent (PS stent, NIR stent, or Terumo stent).

A commercially available stent was immersed in a drug-containing solution for five minutes while the stent is expanded, followed by drying.

(2) Multi-layer-coated Drug-eluting Stent

Preparation of a stent which releases a drug over a long period of time.

(a) Two-layer-coated Drug-eluting Stent

Resin for inner layer: PLA 0010 (polylactic acid) or PLGA 7510 or PLGA 5010 (Wako) (polylactic acid/polyglycolic acid copolymer);

Drug for inner layer: phenolsulfonphthalein, bromosulfophthalein, or a mixture of phenolsulfonphthalein and Evans Blue;

Solvent for inner layer: dichloromethane or cyclohexane;

Resin for outer layer: polyvinyl alcohol or PLA 0010 (polylactic acid);

Drug for outer layer: phenolsulfonphthalein, bromosulfophthalein, or a mixture of phenolsulfonphthalein and Evans Blue;

Solvent for outer layer: dichloromethane or cyclohexane; and

Stent: commercially available coronary stent (PS stent, NIR stent, or Terumo stent).

(b) Three-layer-coated Drug-eluting Stent

Resin for inner layer: PLGA 7510 (polylactic acid/polyglycolic acid copolymer);

Drug for inner layer: phenolsulfonphthalein or bromosulfophthalein;

Solvent for inner layer: dichloromethane or cyclohexane;

Resin for intermediate layer: PLGA 5010 (polylactic acid/polyglycolic acid copolymer), polyphosphoric acid, or silicone;

Resin for outer layer: PLA 0010 (polylactic acid);

Drug for outer layer: phenolsulfonphthalein, bromosulfophthalein, or a mixture of phenolsulfonphthalein and Evans Blue;

Solvent for outer layer: dichloromethane or cyclohexane; and

Stent: commercially available coronary stent (PS stent, NIR stent, or Terumo stent).

(B) Self-absorbable (Biodegradable) Drug-eluting Stent

Stent which disintegrates by itself and disappears, while gradually releasing a drug contained therein.

Stent material: a mixture of PLGA 5010 or PLGA 7510 and polyisoprene;

Drug: phenolsulfonphthalein or bromosulfophthalein; and

Solvent: dichloromethane or cyclohexane.

(C) Photopolymerizable, Self-absorbable Drug-eluting Stent

Partially self-absorbable drug-eluting stent which exhibits flexibility and can be inserted in a compactly folded form. When this stent is inserted into a blood vessel and then irradiated with light having a specific wavelength, the stent is hardened to dilate the blood vessel lumen. This stent can also be employed in a very thin blood vessel.

Stent material: a mixture of any of PLA 0010, PLGA 5010, and PLGA 7510, and urethane dimethacrylate (photopolymerizable resin);

Drug: phenolsulfonphthalein, bromosulfophthalein, or a mixture of either of them and Evans Blue; and

Solvent: dichloromethane or cyclohexane.

Preparation Method

Any of PLA 0010, PLGA 5010, and PLGA 7510, urethane dimethacrylate, phenolsulfonphthalein, and dichloromethane are mixed together in proportions of 1:0.5:0.25:4, to thereby prepare a solution. Subsequently, a polyethylene tube having an outer diameter of 0.2 mm, 0.8 mm, or 1.5 mm is immersed in the solution for five minutes, followed by drying. This procedure is carried out three times. After drying, the tube is removed from the resultant product, to thereby form a stent. When this stent is inserted into a blood vessel, and then irradiated with light transmitted through a quartz fiber, the stent is hardened.

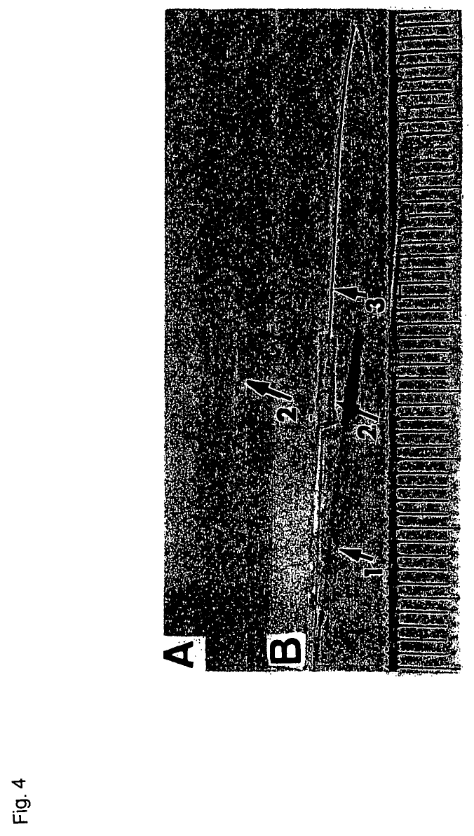

FIG. 4 shows an example of such a stent. FIG. 4A shows a stent having an inner diameter of 0.8 mm, which can be placed in a blood vessel having a diameter of 1 mm or less which prevents a conventional stent from being inserted thereinto, restenosis of which cannot be prevented by means of the conventional stent. FIG. 4B shows a dilation balloon catheter covered with the stent of FIG. 4A. The above-prepared stents other than the stent of FIG. 4 are described in Table 7 which shows the results of drug release test.

(D) Drug Release Test

Each of the above-prepared drug-containing stents was added to a container containing saline (10 mL), the container being placed in a thermostatic chamber maintained at 25° C., and release of the drug was observed. The saline is colored red with phenolsulfonphthalein, or colored violet with bromosulfophthalein. Table 7 shows the degree of release of drugs, the release duration of drugs, and self-disintegration of stents.

TABLE 7
Release of phenolsulfonphthalein from stent and disintegration of resin

| | | Rapid- | | | |
|---|---|---|---|---|---|
| | | release | Release | | Time until initiation of |
| Drug-containing resin, etc. | | property | duration | | self-disintegration of resin |
| A. | Stent coated with drug-containing resin | | | | |
| 1. | Single-layer-coated stent | | | | |
| | Methyl alcohol | +3 | 3 | hours | |
| | Hydroxypropyl alcohol | +3 | 1 | hour | |
| | Polybutyl acrylate | +3 | 5 | days | |
| | Polyvinyl alcohol | +3 | 30 | minutes | |
| | Polyethylene glycol | +3 | 1 | hour | |
| | PLA 0010 | +2 | 7 | days | 7 days |
| | PLGA 5010 | +1 | 12 | days | 30 days |
| | PLGA 7510 | +1 | 15 | days | 28 days |
| | Polyglycolic acid | 0 | 4 | days | |
| | Polyprolactone | 0 | 5 | days | |
| | Polyvinyl acetamide | 0 | 6 | days | |
| | Nippollan 131 | +3 | 3 | days | |
| | Gelatin | +3 | 5 | hours | |
| | Hyaluronic acid | +3 | 6 | hours | |
| 2. | Two-layer-coated stent | | | | |
| | Polybutyl acrylate (In) + Hydroxypropyl | +2 | 6 | days | |
| | alcohol (Out) | | | | |
| | PLGA 7510 (In) + Polybutyl acrylate (Out) | +1 | 14 | days | |
| | Polybutyl acrylate (In) + PLGA 7510 (Out) | +2 | 5 | days | |
| | PLGA 5010 (In) + PLA 0010 (Out) | +2 | 31 | days | 7 days (Out) to |
| | | | | | 35 days (In) |
| | PLGA 7510 (In) + PLA 0010 (Out) | +2 | 60 | days | 8 days (Out) to |
| | | | | | 35 days (In) |
| 3. | Three-layer-coated stent | | | | |
| | PLGA 7510 (In) + Polybutyl | +2 | 24 | days | 7 days (Out) to |
| | acrylate (Inter) + PLA | | | | 30 days (In) |
| | 0010 (Out) | | | | |
| | PLGA 7510 (In) + Silicone (Inter) + PLA | +2 | 23 | days | 30 days |
| | 0010 (Out) | | | | |
| B. | Self-absorbable drug-eluting stent | | | | |
| | PLA 7510 + Polyisoprene | +1 | 15 | days | 25 days |
| C. | Photopolymerizable stent | | | | |
| | Urethane dimethacrylate + PLA 0010 | +2 | 16 | days | 8 days |
| | Urethane dimethacrylate + PLGA 5010 | +1 | 31 | days | 15 days |
| D. | Stent containing two drugs | | | | |
| | Phenolsulfonphthalein + Evans Blue + PLA | +3 | 7 | days | 7 days |
| | 0010 | | | | |

In: inner layer,
Inter: intermediate layer,
Out: outer layer,
+1: weak,
+2: moderate,
+3: strong

(E) Test on Insertion of Drug-eluting Stent into Coronary Artery
(1) Test Method

Nembutal-anesthetized beagle dogs were employed for the test. While each of the animals was ventilated with air, an 8-French guide catheter was inserted through the right common carotid artery, followed by left coronary angiography. Subsequently, a drug-eluting stent (inner layer: PLGA 7510 containing 50% phenolsulfonphthalein, outer layer: PLA 0010 containing 50% phenolsulfonphthalein) was inserted into the left circumflex branch, and then expanded at 9 atm, followed by placement of the stent. After recovery from anesthesia, the animal was bred for one month, and then was again subjected to coronary angiography. The degree of stenosis was measured at the most stenotic site of the stent-inserted portion, and the thus-measured stenosis degree was compared with that in the case of a non-eluting stent group (i.e., a group in which a stent that is not coated with a drug was inserted). Subsequently, the heart was extirpated from the animal, and then subjected to formalin fixation. Thereafter, a microscopic sample of the stent-inserted portion was prepared, followed by staining of vascular smooth muscle β-actin. Stem cells per unit area were counted at the highest-cell-density site of the intima, media, or adventitia of the above-stained sample. Comparison (in terms of stem cell density) was performed between the drug-eluting stent group and the non-eluting stent group (i.e., the group in which a stent that is not coated with a drug was inserted).

(2) Results

Table 8 shows the density of β-actin-positive stem cells. The stem cell density is significantly lower in the drug-eluting stent group. As shown in Table 9, the stenosis degree is also significantly lower in the drug-eluting stent group.

TABLE 8
Density of vascular stem cells in coronary artery with
inserted two-layer-coated drug-eluting stent

| | | Number of |
|---|---|---|
| | Number of | stem cells |
| | samples | (250 × 250 μm²) |
| Intima | | |
| Non-eluting stent group | 6 | 37.8 ± 7.7 |
| Phenolsulfonphthalein group | 6 | 7.1 ± 3.2** |
| Bromosulfophthalein group | 6 | 8.8 ± 2.6** |
| Media | | |
| Non-eluting stent group | 6 | 22.2 ± 7.0 |
| Phenolsulfonphthalein group | 6 | 9.0 ± 4.0* |
| Bromosulfophthalein group | 6 | 8.6 ± 5.0* |
| Adventitia | | |
| Non-eluting stent group | 6 | 40.2 ± 11.0 |
| Phenolsulfonphthalein group | 6 | 16.0 ± 7.1* |
| Bromosulfophthalein group | 6 | 20.3 ± 9.9* |

Inner layer: PLGA 7510,
Outer layer: PLA 0010,
*P < 0.05
**P < 0.05

TABLE 9
Degree of stenosis of coronary artery
with inserted drug-eluting stent

| | Number of | Stenosis |
|---|---|---|
| | samples | degree (%) |
| Non-eluting stent group | 6 | 85.0 ± 6.2 |
| Phenolsulfonphthalein group | 5 | 0 ± 0.0*** |
| Bromosulfophthalein group | 6 | 11.2 ± 3.1** |

### INDUSTRIAL APPLICABILITY

Employment of the drug of the present invention enables effective prevention of vascular restenosis, which would occur as a result of angioplasty or vascular grafting for angina pectoris, myocardial infarction, peripheral vascular occlusion. Also, the drug of the present invention can prevent, for example, stenosis or aneurysm associated with angiitis for which an effective preventive or therapeutic method has not yet been established, the progression and disruption of arteriosclerotic lesion, as well as microvascular ischemic diseases. In addition, the drug of the present invention is envisaged to prevent angiogenic diseases or neural proliferative diseases (e.g., glioma), in which local drug administration is difficult, even when the drug is administered intraarterially or intravenously. Therefore, the drug of the present invention is very useful clinically.

The vascular treatment instrument of the present invention can be placed in a blood vessel over a long period of time. When this instrument is employed for a medical treatment such as angioplasty (e.g., coronary angioplasty, peripheral arteriovenous angioplasty, or cerebral arteriovenous angioplasty) or vascular bypass graft surgery (coronary bypass graft surgery or peripheral arteriovenous bypass graft surgery), occurrence of vascular restenosis can be effectively prevented.

1. A drug for preventing invasion of bone-marrow-derived vascular stem cells or inflammatory cells into blood vessel walls, the drug comprising a phthalein dye compound as an active ingredient.

2. A drug for preventing vascular restenosis, the drug containing a phthalein dye compound as an active ingredient.

3. A preventive or therapeutic drug for angiitis or myocarditis, the drug comprising a phthalein dye compound as an active ingredient.

4. The preventive or therapeutic drug according to claim 3, wherein the angiitis is Kawasaki disease, aortitis syndrome, giant cell arteritis, arteritis associated with ankylosing arthritis, or arteriosclerotic lesion (plaque).

5. A preventive or therapeutic drug for a microvascular ischemic disease, the drug comprising a phthalein dye compound as an active ingredient.

6. A preventive or therapeutic drug for a vascular disease, the drug comprising a phthalein dye compound as an active ingredient.

7. The preventive or therapeutic drug according to claim 6, wherein the vascular disease is moyamoya disease or diabetic retinopathy.

8. A preventive or therapeutic drug for a proliferative disease caused by cells derived from neural stem cells, the drug comprising a phthalein dye compound as an active ingredient.

9. A drug for preventing graft rejection, the drug comprising a phthalein dye compound as an active ingredient.

10. A preventive or therapeutic drug for arteriosclerosis associated with organ transplantation, the drug comprising a phthalein dye compound as an active ingredient.

11. The drug according to any one of claims 1 through 10, which further contains a sulfonic acid-based azo dye.

12. The drug according to any one of claims 1 through 10, which further contains an immunosuppressive drug, an immunomodulator, a collagen synthesis inhibitor, and/or an anticancer drug, etc.

13. The drug according to any one of claims 1 through 12, wherein the phthalein dye compound is phenolsulfonphthalein or bromosulfophthalein.

14. A vascular treatment instrument coated with a resin comprising a phthalein dye compound.

15. A vascular treatment instrument prepared from a resin comprising a phthalein dye compound.

16. The vascular treatment instrument according to claim 14 or 15, which is an artificial blood vessel, a stent, or a stent graft.

17. The vascular treatment instrument according to any one of claims 14 through 16, which is formed of a material selected from among a carbon nanosheet, a self-absorbable material, or a photopolymerizable material.

18. The vascular treatment instrument according to any one of claims 14 through 17, wherein the phthalein dye compound is phenolsulfonphthalein or bromosulfophthalein.

19. A percutaneous transluminal arteriovenous angioplasty or an arteriovenous bypass graft surgery, characterized by employing a vascular treatment instrument coated with a resin containing a phthalein dye compound, or a vascular treatment instrument prepared from the resin.

20. A method of preventing vascular restenosis, characterized by comprising subjecting a patient to a medical treatment employing a vascular treatment instrument coated with a resin containing a phthalein dye compound, or a vascular treatment instrument prepared from the resin.

21. A method of treating an inflammatory angiocardiac disease, characterized by comprising administering a phthalein dye compound to a patient in need thereof.
